(19) United States
(12) Patent Application Publication
(10) Pub. No.: US 2020/0138439 A1
(43) Pub. Date: May 7, 2020

(54) Implant for Injured Nerve Tissue Prosthetics, Method of Surgical Treatment for Injured Nerve Tissue and Use of Porous Polytetrafluorethylene

(71) Applicant: Anatoli D. DOSTA, Minsk (BY)

(72) Inventor: Anatoli D. DOSTA, Minsk (BY)

(21) Appl. No.: 16/712,379

(22) Filed: Dec. 12, 2019

Related U.S. Application Data
(63) Continuation of application No. PCT/BY2017/000017, filed on Sep. 26, 2017.

(30) Foreign Application Priority Data
Jun. 13, 2017 (BY) A20170215

(51) Int. Cl. A61B 17/11; A61L 27/16; A61L 27/56

(52) U.S. Cl. CPC A61B 17/1128; A61L 2430/32; A61L 27/56; A61L 27/16

(57) ABSTRACT

An implant is suitable for treatment for nerve tissue injuries of various types in any period of the severe injury to the nerve tissue, in particular, of the spinal cord, immediately after relief of disturbed vital functions for the early and stable restoration of its conduction in the acute period, prevention from or reduction of the demyelination processes. The technical result is to ensure the possibility to restore the injured nerve tissue in volume. The implant is the body made from porous material, such as the porous PTFE having three-dimensional structure containing the open through pores and dead-ended pores uniformly distributed over inner surfaces of the open pores and connected with the inner surfaces; pore sizes are randomly distributed within the range of 150-300 μm. The method of treatment for nerve tissue injuries and use of the porous PTFE for manufacture of the implant are also claimed.

### RELATED APPLICATIONS

This application is a Continuation application of International Application PCT/BY2017/000017, filed on Sep. 26, 2017, which in turn claims priority to Belarusian Patent Application BY a20170215, filed Jun. 13, 2017, both of which are incorporated herein by reference in their entirety.

### FIELD OF THE INVENTION

The invention relates to medicine and may be used in neurosurgery, traumatology, neurology, rehabilitation.

### BACKGROUND OF THE INVENTION

The known method of treatment for the sequelae of a traumatic injury to the spinal cord is to transplant intercostal nerves into the injured spinal cord [Yumashev G. S., Ziablov V. I., Korzh A. A. et al.//Orthopedist, Traumatol.-1989-1. P. 71-71]. However, such method has the insignificant clinical effect. The axons in the central nervous system (further—CNS) appeared to be able to regenerate inside such implants, but unable to grow outside such implants, in order to restore connections with other CNS neurons; regenerating neurons “stick” inside a implant as a result of formation of a collagen scar.

There is one more known method of introduction of embryonal tissue bits between the central and peripheral ends of the injured spinal cord [Patent of Russia No. 2195941, publication 10.01.20031]. It cannot be considered sufficient, as the experimental studies have proven that with transplantation of an embryonal spinal cord fragment, the overlying axons grow out to the length of an implant, at the best, i.e. by 1-1.5 cm. The recipient axons do not grow more distally that the implant, they stick in the collagen scar.

Another known method of treatment for the sequelae of the spinal cord injury is to place the container, containing Schwann's cells in the special gel, between the ends of the injured spinal cord. The Schwann's cells obtained from explants of human or rat nerves are cultivated, and their amount increases significantly. Then the cells are placed in the matrix filling the semipermeable tubes, and they, in turn, place between the cut ends of the spinal cord. The result of most transplantations of Schwann's cells is the regeneration of most CNS axons, their growing through the implant, however, the axons were unable to leave the microenvironment of the Schwann's cells, in order to introduce again in the depth of the spinal cord tissues and form new interneuron connections [Patent of China No. 101653366, publication 24 Feb. 2010].

Thus, the above-mentioned known methods may not be used for the effective restoration of the spinal cord function because the obstacles, i.e. collagen (connective-tissue) scar, cannot be overcome on the way of axon growth. Axons are unable to grow outside implants, in order to restore the connections with other CNS neurons; regenerating neurons “stick” inside an implant.

In addition, human tissue fragments were used as implants in the methods described, and this can result in both foreign body reaction and increased risk of the infection carry.

The implant and the method of treatment for spinal cord injuries under Patent of USA No. 7147647, publication 12 Dec. 2006 describing the implant as a porous titanium tube which inner and outer surface has one or several porous layers, with pore diameter of 1-3 μm and depth upto 0.5 μm, is the nearest Prior Art reference. The tube diameter depends on the diameter of the nerve subject to treatment.

The method of treatment is to place an injured nerve inside the claimed tube.

The disadvantage of this technical solution is the fact that the axon growth area is the porous layer of the inner surface of the tube only, thus, determining the limited number of nervous connections restored.

Additionally, in order to be placed in the implant described, the injured nerve should be selected from the surrounding tissues, and this is possible for far from all areas of the human nerve tissue. In particular, the described implant is inapplicable to the spinal cord, as well as for other areas in any period of the severe injury immediately after relief of disturbed of vital functions what should contribute to the early and stable restoration of the spinal cord conduction in the acute period, prevent from or reduce the demyelination processes.

### SUMMARY OF THE INVENTION

The aim of the claimed group of inventions is to create the implant suitable for treatment for nerve tissue injuries of various types, in any period of the nerve tissue severe injury, in particular, of the spinal cord, immediately after relief of disturbed vital functions for the early and stable restoration of nerve tissue conduction in the acute period, prevention from or reduction of the demyelination processes. The technical result enabling to solve this aim—ensuring the possibility to restore the injured nerve tissue in volume.

The aim assigned is performed in the implant for the injured nerve tissue prosthetics which implant presents the body made from the porous material: the porous material is the porous polytetrafluorethylene (further—PTFE) having the three-dimensional structure containing the open through pores and dead-ended pores uniformly distributed over inner surfaces of the open pores and connected with the inner surfaces; pore sizes are randomly distributed within the range of 150-300 μm.

The nerve tissue may be the spinal cord tissue or the acoustic nerve or the optic nerve.

If the nerve tissue injury is destruction of the nerve tissue area or slight tear of the nerve tissue or the collagen scar subject to excision, the implant is preferably made in the form of a plate for substitution of the missing nerve tissue.

If the nerve tissue injury is necrosis of the nerve tissue area, the implant may be made in the form of a split coupling, in order to overlap the necrotic nerve tissue area.

The aim assigned is also performed in the method of the surgical treatment for the injured nerve tissue by placement of the porous material in the injure area, due to the fact that the porous material being used is the porous PTFE having the three-dimensional structure containing the open through pores and dead-ended pores uniformly distributed over inner surfaces of the open pores and connected with the inner surfaces; pore sizes are randomly distributed within the range of 150-300 μm.

The nerve tissue may be the spinal cord tissue or the acoustic nerve or the optic nerve.

If the nerve tissue injury is destruction of the nerve tissue area or slight tear of the nerve tissue or the collagen scar subject to excision, the implant is preferably made in the form of a plate and placed on the place of the missing nerve tissue fragment or in the area of the collagen scar excised.

If the nerve tissue injury is necrosis of the nerve tissue area, the implant is preferably made in the form of a split coupling and placed over the necrotic nerve tissue area.

The assigned aim is also performed due to use of the porous PTFE having the three-dimensional structure containing the open through pores and dead-ended pores uniformly distributed over inner surfaces of the open pores and connected with the inner surfaces; pore sizes are randomly distributed within the range of 150-300 μm, for manufacture of the implant for the injured nerve tissue prosthetics.

### BRIEF DESCRIPTION OF THE DRAWINGS

This invention is shown as an example on the following unlimiting drawings.

A schematic view of the first variant of the claimed implant is shown in FIG. 1.

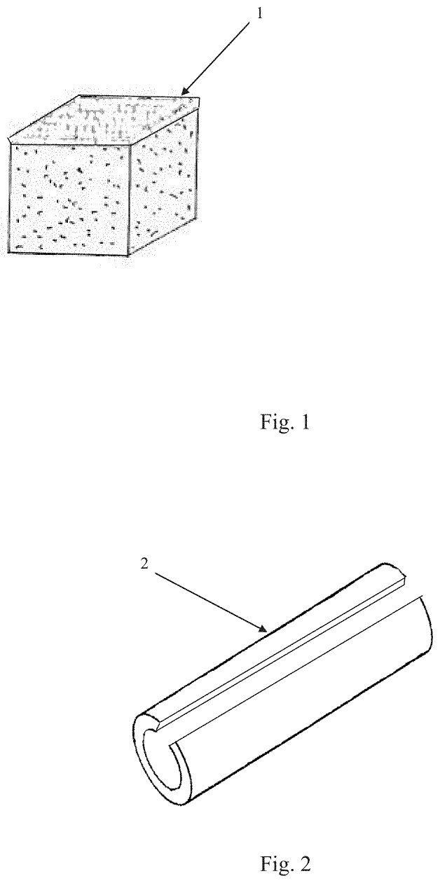

A schematic view of the second variant of the claimed implant is shown in FIG. 2.

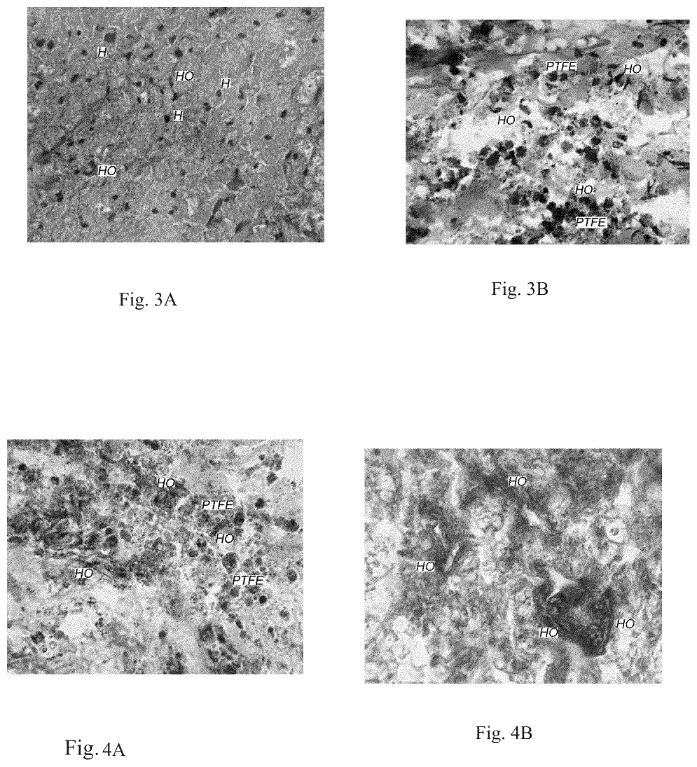

Images of spinal cord sections for Example 1 are shown in FIGS. 3A-3B;

Images of spinal cord sections for Example 1 are shown in FIGS. 4A-4B;

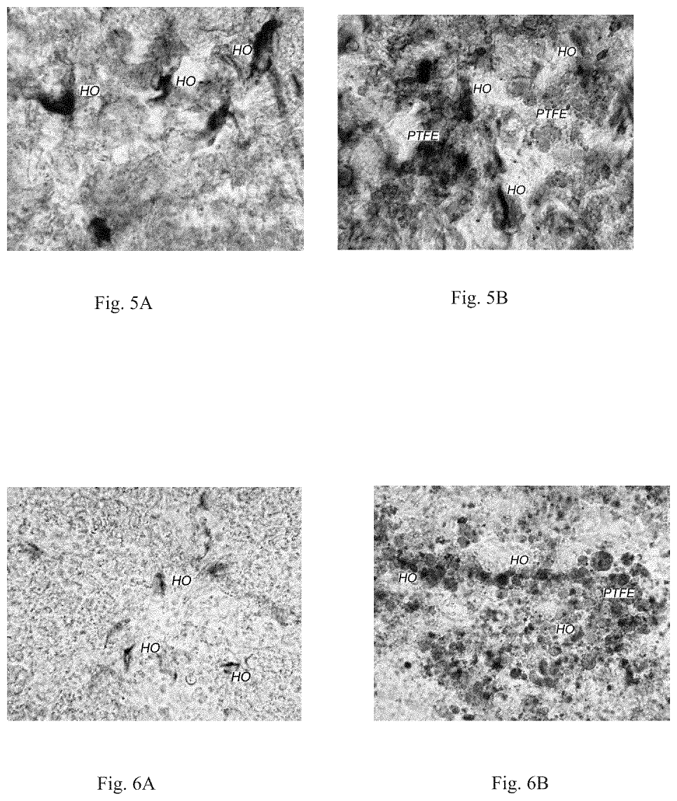

Images of spinal cord sections for Example 1 are shown in FIGS. 5A-5B;

Images of spinal cord sections for Example 1 are shown in FIGS. 6A-6B;

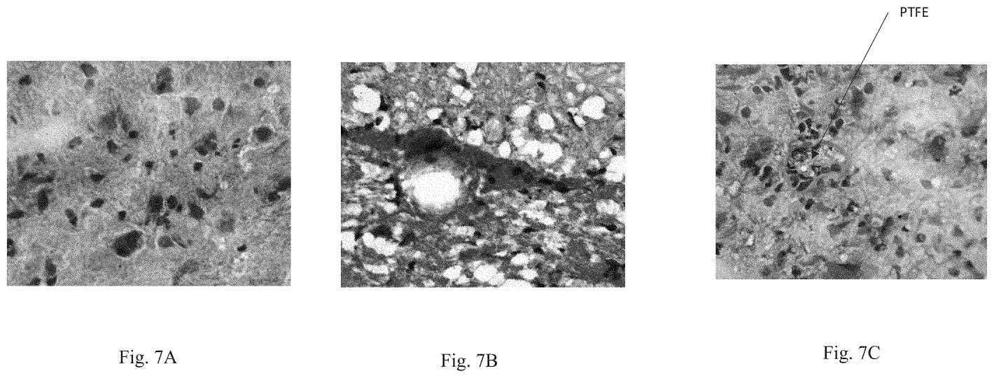

Images of spinal cord sections for Example 2 are shown in FIGS. 7A-7C;

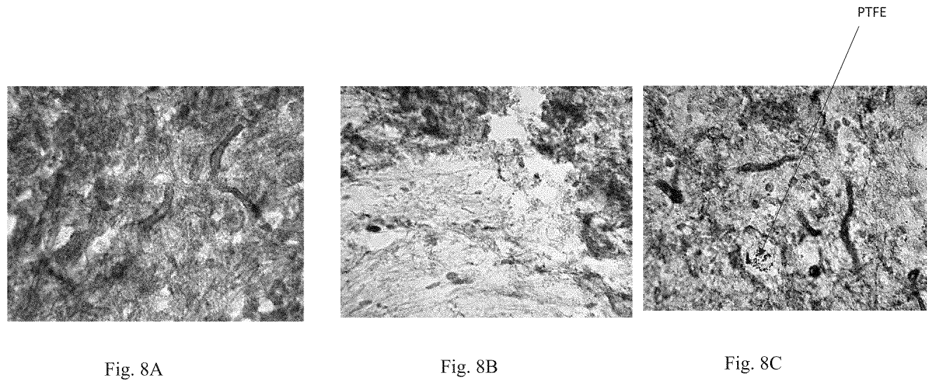

Images of spinal cord sections for Example 2 are shown in FIGS. 8A-8C;

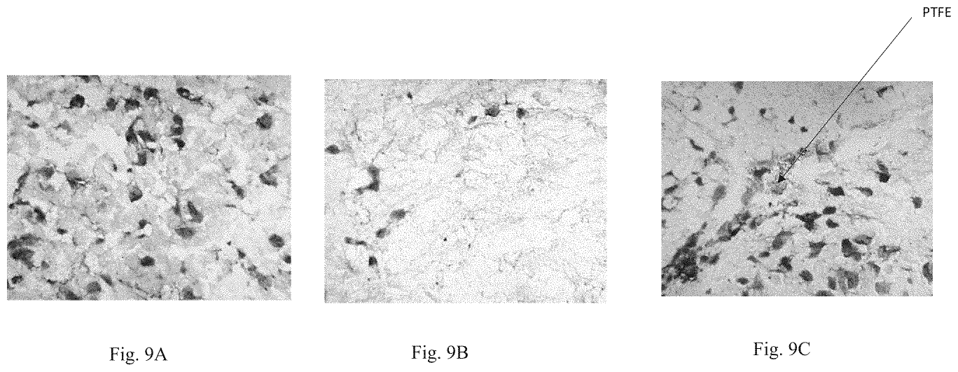

Images of spinal cord sections for Example 2 are shown in FIGS. 9A-9C;

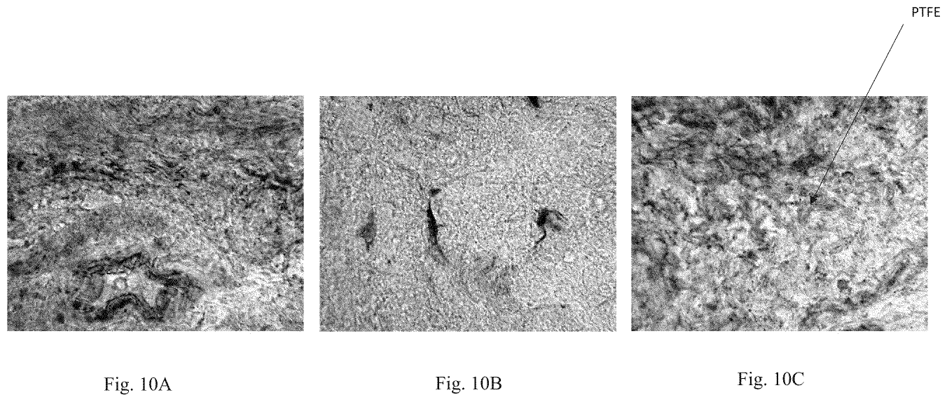

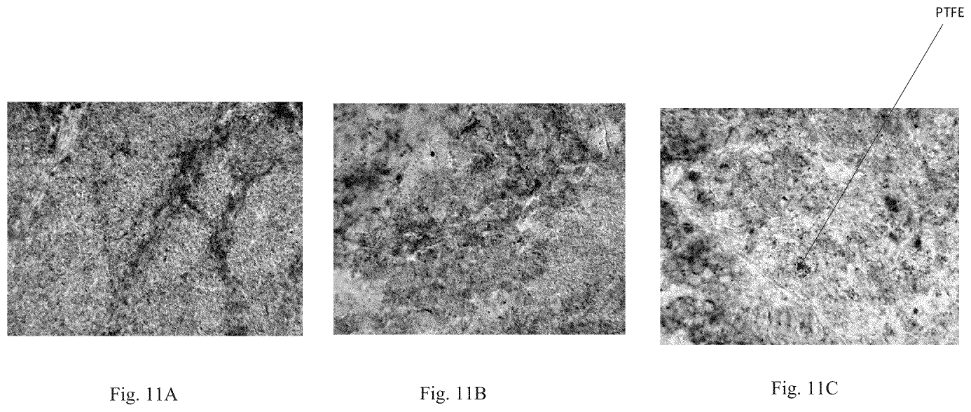

Images of spinal cord sections for Example 2 are shown in FIGS. 10A-10C; and

Images of spinal cord sections for Example 2 are shown in FIGS. 11A-11C.

The claimed implant may be manufactured by the method, for example, described in Patent of Belarus No. 10325, publication 28 Feb. 2008. The porous PTFE implant is manufactured by mixing of raw material granules with pore-former (common salt) granules, compression of the mixture obtained, wash-out of common salt from the obtained porous blank and its further sintering. The complex structure of pores is caused, in such case, by the comminuted form of pore-former granules. The sizes of the dead-ended pores are determined by sizes of pore-former small-fraction grains and sizes of the open through pores—by sizes of pore-former large-fraction grains.

### DETAILED DESCRIPTION OF THE PREFERRED EMBODIMENTS

The claimed method of the surgical treatment for the spinal cord injury is performed, for example, as follows.

Perform nuclear magnetic resonance tomography (further—NMR tomography) for the spinal cord for the patient with the spinal cord injury.

Determine the localization and size of the spinal cord defect, availability of cysts and commissures.

On the grounds of these determinations, cut the plate 1 of the spinal cord implant (FIG. 1) with the target size and shape from the pre-manufactured porous PTFE plate, sterilize and store in the sterile packing.

The implant porous structure may be saturated with drugs or the nerve tissue growth stimulator.

Perform the typical laminectomy for the patient in the lateral recumbent position; open the pachymeninx.

Make the meningomyelolysis with 3.5× magnification; expose the distal and proximal ends of the injured spinal cord area.

Excise the formed collagen (connective-tissue) scar

Place the prepared implant plate 1 in such a way as to fill the space between the ends of the patient's injured spinal cord area.

Suture the operative site layer-by-layer, tightly.

The claimed method of the surgical treatment for the necrotic injury of, for example, the acoustic nerve is performed, for example, as follows.

In such case, choose the implant in the form of a split coupling 2 (FIG. 2).

In the course of the operation expose the necrotic area of the nerve in such a way as to have access to non-necrotic areas;

choose the diameter of implant split coupling 2: the implant should closely adjoin the non-necrotic areas of the nerve;

chose the length of the implant split coupling 2: it should be longer than the injured area.

Open the implant split coupling 2 and place it in such a way as to overlap the necrotic area of the nerve, with non-necrotic areas of the nerve covered.

Suture the operative wound.

The animal studies, as described in below examples, have been carried out, in order to check the workability and effectiveness of the inventions claimed.

### Example 1

The operation of half-transsection of the dog's spinal cord in the region of T11 segment was made with the further implantation of the implant in the form of a porous PTFE plate, according to the disclosure, in the injury area. The restoration of the motor activity of the experimental animal was recorded.

Three months after the operation, the spinal cord fragments in the place of contact with the implant as well as the spinal cord fragment of the intact animal were removed. FIGS. 3-6 show (×400) the results of the examination of the spinal cord of the intact (control) dog (a) and experimental dog (b).

Materials and Methods

The material of the study is the dog spinal cord fragments in the places of contact with the grafts. After removal the test material was placed on ice.

The sections were divided in groups depending on morphological examinations.

Series 1. Stain with haematoxylin-eosin (general histology).

Series 2. Nissl stain (visualization of nerve tissue elements).

The micro-preparations were studies and micro-photos were made with MPV-2 light microscope (made by Leitz, Germany) with Leica digital camera with the software and computer.

The morphological changes were evaluated at the light-optic level.

The databases with the results of morphological examinations were formed with the use of MS Excel. The statistical analysis of the obtained results was made with the STATISTICA 6.1 program (SrtatSoft).

FIG. 3A shows the section of the spinal cord area in the region of the thoracic vertebra (T11) of the intact dog; FIG. 3A shows the section of the dog spinal cord area 3 months after the half-transsection and destruction of the thoracic vertebra (T11) and placement of the PTFE implant at an angle of 45°. Treatment with haematoxylin-eosin (×400).

In FIG. 3B, one can observe the rearrangement of the spinal cord area structure in the places of PTFE placement. The nerve cell appendages grow into the implant pores, proving the restored nerve impulse conduction in the transsection region. No hypertrophy of the connective tissue or formation of a coarse collagen scar was noted.

Determination of acetylcholinesterase (ACE) activity allows to judge on availability of the acetylcholine mediator which is characteristic of the cholinergic (parasympathetic) nature of nerve elements. The final product of the reaction running with participation of the acetylcholinesterase enzyme was determined in the form of copper ferrocyanide sediments staining the cholinergic nerve masses—nerve fibres and endings, into the brown colour (in FIGS. 4a and 4b, HO—nerve cell appendages, showed in black).

The cholinergic innervation in the region of the spinal cord injury restores slower, as proved by lower values of ACE activity in the nerve fibres regenerating in the PTFE implanted in the injured spinal cord area, as compared to the intact ones. The reduced activity of acetylcholinesterase is caused by appearance, in the injury sites, of regenerating nerve cell appendages which diameter is significantly smaller than in the intact sites.

The histochemical methods of detection of the cytoplasmic enzymes characterizing the metabolic activity of cells: succinate and lactate dehydrogenases (SDG and LDG), were used in the experiment. The availability of the enzymes in the dog spinal cord is indicated by the dark blue sediment of formazan which is formed with the reduction of tetrazolium salts (main localization place—the internal membrane of mitochondria and divergent cristae, sarcoplasmic reticulum). The activity of enzymes was evaluated under the optical density of the reaction product in the cell cytoplasm (formazan) by means of Image J data processing computer program, 100 cells in each of 5 sections were considered.

FIGS. 5A and 5B show the detection of lactate dehydrogenase in the spinal cord neurons and nerve cell appendages (HO—nerve cell appendages, showed in black). The comparative analysis of the histochemical data obtained with measurement of mean values of LDG activity in the spinal cord nerve cell appendages in the injury region with the PTFE and in the regions above and below the injury site, showed the significant increase of LDG enzyme activity in the injury region by 54% and 50%, respectively. This reaction promotes the acceleration of restoring cell appendages in the nerve tissue.

FIGS. 6A and 6B show the detection of succinate dehydrogenase in the spinal cord neurons and nerve cell appendages (HO—nerve cell appendages, showed in black).

The comparative analysis of the histochemical data obtained with measurement of mean values of SDG activity in the spinal cord nerve cell appendages in the injury region with the PTFE and in the intact regions above and below the injury site, showed the significant increase of the enzyme activity in the injury region by 57% and 52%, respectively.

Based on the histological (stain with haematoxylin and eosin), neurohistological (Nissl stain) and histochemical (detection of ACE, LDG and SDG activity) examinations, one may conclude that:

The rearrangement of the structure of spinal cord area under test was observed in the places of PTFE placement (FIG. 3B). The nerve cell appendages grew into the implant pores throughout the volume of the PTFE implanted, proving the restored nerve impulse conduction in the transsection region. No hypertrophy of the connective-tissue (collagen) scar was noted.

The spinal cord neuron cell appendages regenerate actively in the region of the spinal cord injury, into the pore of the implanted PTFE throughout the volume in the side of the adjoining intact regions of the spinal cord.

The neuron cell appendages regenerating in the PTFE implanted in the injured region of the spinal cord, restore its functional activity, as showed by the significant increase of the activity values of the energy metabolism enzymes—LDG and SDG in regenerating nerve cell appendages

No significant differences were found in the percentage of viable cells in the dog spinal cord samples without the half-transsection of the spinal cord or after the PTFE implant placement in the region of the experimental injury.

No significant difference was detected in the course of the calculation of histochemical values of CD90 (stem cell marker) expression in the spinal cord samples from the intact dog and the dog after the PTFE implant placement in the region of the experimental injury.

### Example 2

The spinal cord of rats was the object of the study; rats were divided into 3 groups: group 1—intact rats (control), group 2—the rats which were subjected to half-transsection of the spinal cord, group 3—the rats which were subjected to half-transsection of the spinal cord with further implantation of the PTFE in the injury region. Observation period—2 months.

The works was performed with the use of the histological (stain with haematoxylin and eosin), neurohistological (Nissl stain) and histochemical (detection of acetylcholinesterase, succinate and lactate dehydrogenases (ACE, LDG and SDG) activity examinations.

The frozen sections of the spinal cord were stained with haematoxylin and eosin and toluidine blue, and then they were examined at the light-optic level.

FIG. 7A shows the section of the spinal cord area in the region of the thoracic vertebra (T11) of the intact rat. Treatment with haematoxylin-eosin (×400).

FIG. 7B shows the section of the rat spinal cord area after the half-trans section and destruction of the thoracic vertebra (T11) without the implant placement. Treatment with haematoxylin-eosin (×400). Along the edge of the scar tissue one can observe the formation of the glial capsule (intensive red colour (black colour—in the drawing), course connective-tissue (collagen) scar) which wall is formed by glial cells, predominantly, astrocytes, locating in the form of the multilayer shaft. The glial cells, as detected in adjoining regions of the spinal cord, undergo dystrophic changes. Hemodynamic disorders are found in the adjoining areas of the spinal cord, they are the result of the necrobiotic changes in blood vessel walls, entry of the blood liquid fraction to the circumvascular space and development of pericapillary oedema. Vacuolization and cytoplasm swelling, destruction of some cells (white hollows) are noted.

FIG. 7C shows the section of the rat spinal cord area after the half-trans section and destruction of the thoracic vertebra (T11) and placement of the PTFE implant at an angle of 45°. Treatment with haematoxylin-eosin (×400). A light-grey area—PTFE.

A friable connective-tissue (collagen) scar is found in the test region; there are newly formed blood capillaries in the depth of the collagen scar, proving the active angiogenesis in the collagen scar tissue. One can detect clusters of glial cells—astrocytes locating diffusely and without formation of the glial demarcation line along the collagen scar periphery preventing from regeneration of the nerve tissue, as well as multiple neurons with long branching appendages indicative of the regeneration activity of nerve fibres and restoration of the nerve conduction in the injury region. Treatment with haematoxylin-eosin (×400).

Determination of acetylcholinesterase (ACE) activity allows to judge on availability of the acetylcholine mediator which is characteristic of the cholinergic (parasympathetic) nature of nerve elements. The final product of the reaction running with participation of the acetylcholinesterase enzyme, was determined in the form of copper ferrocyanide sediments staining the cholinergic nerve masses—nerve fibres and cell appendages, into the brown colour.

FIG. 8A shows the section of the spinal cord area in the region of the thoracic vertebra (T11) of the intact rat.

FIG. 8B shows the section of the rat spinal cord area after the half-trans section and destruction of the thoracic vertebra (T11) without the implant placement. The rough destruction of nerve fibres and non-uniform accumulation of the enzyme in nerve cells, up to absence, are noted. The acetylcholinesterase activity is reduced.

FIG. 8C shows the section of the rat spinal cord area after the half-trans section and destruction of the thoracic vertebra (T11) and placement of the PTFE implant at an angle of 45°. Gray-black colour—PTFE. The activity of ACE enzyme in regenerating nerve fibres is higher than in the group of the rats without the implant placement.

FIGS. 9A-9C show the rat spinal cord cross-sections which were Nissl stained (visualization of nerve tissue elements only, intensive blue colour (black colour—in the drawing)
neuron bodies and cell appendages) (×400).

FIG. 9A shows the section of the spinal cord area in the region of the thoracic vertebra (T11) of the intact rat.

FIG. 9B shows the section of the rat spinal cord area after the half-trans section and destruction of the thoracic vertebra (T11) without the implant placement. The regeneration of single nerve cell appendages against the wide growth of the connective tissue.

FIG. 9C shows the section of the rat spinal cord area after the half-trans section and destruction of the thoracic vertebra (T11) and placement of the PTFE implant at an angle of 45°. A light-grey area—PTFE. The active regeneration of nerve cell appendages into the injury area is observed.

The histochemical methods of detection of the cytoplasmic enzymes characterizing the metabolic activity of cells: succinate and lactate dehydrogenases (SDG and LDG), were used in the experiment. The availability of the enzymes in the rat spinal cord is indicated by the dark blue sediment of formazan which is formed with the reduction of tetrazolium salts (main localization place—the internal membrane of mitochondria and divergent cristae, sarcoplasmic reticulum). The activity of enzymes was evaluated under the optical density of the reaction product in the cell cytoplasm (formazan) by means of Image J data processing computer program, 100 cells in each of 5 sections were considered.

FIGS. 10A-10C show the rat spinal cord cross-sections with visualization of the LDG activity. The dark blue colour is indicative of the presence of the enzyme (black colour—in the drawing). (×400).

FIG. 10A shows the section of the spinal cord area in the region of the thoracic vertebra (T11) of the intact rat.

FIG. 10B shows the section of the rat spinal cord area after the half-transsection and destruction of the thoracic vertebra (T11) without the implant placement. The LDG activity in the spinal cord nerve cell appendages with the half-transsection of the spinal cord without the PTFE implantation was M±m=89.89±17.64 (s.u.), i.e. by 6.0% lower than the values of the control animals and by 11.0% lower than the activity of the LDG enzyme in the rats with the PTFE implanted in the region of the spinal cord transsection.

FIG. 10C shows the section of the rat spinal cord area after the half-transsection and destruction of the thoracic vertebra (T11) and placement of the PTFE implant at an angle of 45°. A light-grey area—PTFE. The increase in the LDG activity results in the activation of the glycolytic processes and, consequently, in the intensification of the reparative processes aimed at the restoration of the injured area of the spinal cord.

FIGS. 11A-11C show the rat spinal cord cross-sections with visualization of the SDG activity. The dark blue colour is indicative of the presence of the enzyme (black colour—in the drawing). (×400).

FIG. 11A shows the section of the spinal cord area in the region of the thoracic vertebra (T11) of the intact rat.

FIG. 11B shows the section of the rat spinal cord area after the half-transsection and destruction of the thoracic vertebra (T11) without the implant placement. The reduced SDG activity in the regenerating nerve fibres of the spinal cord in the region of the connective-tissue (collagen) scar is indicative of the inhibition of the oxidation-reduction processes in the Krebs cycle and reduction of the energy metabolism level in the regenerating nerve tissue. The SDG activity in the spinal cord nerve cell appendages with the half-transsection of the spinal cord without the PTFE implantation was M±m=87.47±19.22 (s.u.), i.e. by 24.78% lower than the values of the control animals and by 15.5% lower than the activity of the DDG enzyme in the rats with the PTFE implanted in the region of the spinal cord transsection.

FIG. 11C shows the section of the rat spinal cord area after the half-transsection and destruction of the thoracic vertebra (T11) and placement of the PTFE implant at an angle of 45°. A light-grey area—PTFE.

Use of the inventions claimed allows:

1. To transplant in any period of the severe injury to the nerve immediately after relief of disturbed vital functions what contributes to the early and stable restoration of its conduction in the acute period, prevents from or reduces the demyelination processes. The restoration of the spinal cord function eliminates the harmful consequences of the prolonged inactive state, and this is of high psycho-emotional and socio-economic importance for patients and their relatives.

2. To reduce the disability because of the severe vertebral-cerebrospinal injury.

3. To improve the quality of life of the persons suffered from the severe injury of the spinal cord.

Thus, the present inventions provide with the possibility to restore the injured nerve tissue in volume, and this fact, in turn, determines the suitability of the claimed implant for treatment for nerve tissue injuries of various types, in any period of the severe injury to the nerve tissue, in particular, of the spinal cord, immediately after relief of disturbed vital functions for the early and stable restoration of its conduction in the acute period, prevention from or reduction of the demyelination processes.

What is claimed is:

1. An implant for injured nerve tissue prosthetics, the implant comprising a body made from a porous material the porous material being a porous PTFE having a three-dimensional structure comprising open through pores and dead-ended pores uniformly distributed over inner surfaces of the open pores and connected with the inner surfaces, wherein pore sizes are randomly distributed within the range of 150-300 μm.

2. The implant according to claim 1, wherein the nerve tissue is spinal cord tissue or an acoustic nerve or an optic nerve.

3. The implant according to claim 1, a nerve tissue injury is destruction of a nerve tissue area or slight tear of the nerve tissue, and wherein the implant is made in the form of a plate for substitution of a missing nerve tissue.

4. The implant according to claim 1, wherein a nerve tissue injury is necrosis of a nerve tissue area, and wherein the implant is made in a form of a split coupling to overlap a necrotic nerve tissue area.

5. A method of surgical treatment of injured nerve tissue via placement of an implant comprising a body of a porous material in the injure area, the porous material being the porous PTFE having a three-dimensional structure comprising open through pores and dead-ended pores uniformly distributed over inner surfaces of the open pores and connected with the inner surfaces, wherein pore sizes are randomly distributed within the range of 150-300 μm.

6. The method according to claim 5, wherein the nerve tissue is a spinal cord tissue or an acoustic nerve or an optic nerve.

7. The method according to claim 5, wherein the nerve tissue injury is destruction of a nerve tissue fragment or slight tear of the nerve tissue, and wherein the implant is made in the form of a plate and placed on a place of the missing nerve tissue fragment.

8. The method according to claim 5, wherein a nerve tissue injury is necrosis of a nerve tissue area, and wherein the implant is made in a form of a split coupling and placed over a necrotic nerve tissue area.

9. A method of manufacturing an implant for an injured nerve tissue prosthetics comprising using a porous PTFE three-dimensional structure comprising an open through pores and dead-ended pores uniformly distributed over inner surfaces of the open pores and connected with the inner surfaces, whereinpore sizes are randomly distributed within the range of 150-300 μm.
